(19) United States
(12) Patent Application Publication
(10) Pub. No.: US 2015/0025139 A1
(43) Pub. Date: Jan. 22, 2015

(54) INGENOL MEBUTATE IN COMBINATION WITH CRYOTHERAPY FOR THE TREATMENT OF ACTINIC KERATOSIS

(71) Applicant: LEO Laboratories Limited, Dublin 12 (IE)

(72) Inventor: Torsten Skov, Ballerup (DK)

(21) Appl. No.: 14/444,787

(22) Filed: Jul. 28, 2014

Related U.S. Application Data
(63) Continuation of application No. PCT/EP13/51435, filed on Jan. 25, 2013.
(60) Provisional application No. 61/753,779, filed on Jan. 17, 2013.
(60) Provisional application No. 61/591,093, filed on Jan. 26, 2012.

(51) Int. Cl. A61K 31/22; A61B 18/02; A61K 9/00

(52) U.S. Cl. CPC A61K 31/22; A61K 9/0014; A61B 18/02; A61B 2018/00452
USPC 514/511

(57) ABSTRACT

The invention relates to the treatment of actinic keratosis (AK) lesions using sequential cryotherapy and field treatment with ingenol mebutate (e.g., PEP005 Gel).

### FIELD OF THE INVENTION

The invention relates to the treatment of actinic keratosis (AK) lesions using sequential cryotherapy and field treatment with ingenol mebutate (e.g., PEP005 Gel).

### BACKGROUND OF THE INVENTION

Actinic keratosis (AK) is a common skin condition visible as thickened, cornified, scaly lesions and characterised histologically by atypical epithelial proliferation. Actinic keratoses usually develop on areas that are frequently exposed to the sun (e.g., face, ears, lips, scalp, neck, forearms, and back of the hands). Patients with AK often express embarrassment, worry, and irritation related to the change in appearance of their skin and unsightly nature of the lesions. In addition to the emotional strain, AK lesions can be painful and easily traumatised causing bleeding.

It is estimated that AK occurs in 11-50% of the population aged 40 and older in the US and Australia. In Europe the prevalence rate is from 11-25% for people aged 40 or older. Patients with AK tend to have Fitzpatrick type I or II skin (fair skin) which burns and does not tan.

In the context of AK, field cancerisation is characterised by the epithelial surface of the photodamaged area being susceptible to the development of additional AKs or a malignancy. This is evident by the presence of multiple subclinical and clinically visible AK lesions as well as multifocal preneoplastic changes with genetic mutations. There is also increasing evidence that AK represents squamous cell carcinoma (SCC) in situ in its earliest stages. If left untreated, AK may progress to SCC, with significant morbidity and death.

Ingenol mebutate is an ingenol derivative extracted from Euphorbia peplus (E. peplus), a member of the Spurge family. Ingenol mebutate was identified as the principal active component responsible for the selective cytotoxic effects of E. peplus sap, based on its antitumour effects both in vitro and in vivo. Ingenol mebutate is distinguished from current therapeutic options by a substantially shorter duration of treatment (2 to 3 days) compared to approved topical AK products.

In the U.S., cryotherapy is the most common form of treatment for AK. Although cryotherapy is a standard therapy for AK, treatment duration with cryotherapy is not standardised and this is reflected in a wide range of efficacy results. Following a single freeze session, lesion response rates were 70-90% at 3 months of follow-up (Freeman et. al, J. Dermatolog Treat. 2003; 14(2):99-106, Hauschild et. al, Br. J. Dermatol. 2009, 160(5): 1066-1074 and Szeimies et. al. Br. J. Dermatol. 2010; 162(2): 410-414). However, many patients have several AKs and a patient's likelihood of being cleared of all AKs is also dependent on the number of AKs treated. Unfortunately, only a few reports exist about patient clearance rates and they are even more difficult to interpret. Thai et al. Int. J. Dermatol, 2004; 43(9): 687-692 reported a patient response rate of 57% at 3 months (mild to moderate AKs on the face and scalp, 4.7 lesions per patient, 1 freeze cycle, mean freeze time 13 second). Krawchenko et al. (Br. J. Dermatol. 2007; 157 Suppl 2: 34-40) used an aggressive cryotherapy regimen (2 treatments 2 weeks apart, 20-40 seconds freeze each time, 7.9 lesions per patient) and obtained a patient complete clearance rate of 68% at 6 weeks. Patient response rates around 30% have also been reported (Tan et. al., J. Cutan. Med. Surg. 2007 November-December; 11(6):195-201 and Jorizzo et. al., J. Drugs Dermatol. 2010; 9(9): 1101-1108).

Data on the duration of clearance/recurrence rates are scarce. In the Krawchenko et al. (see above) trial discussed above, a clearance of 4% was reported at 12 months, substantially less than the 68% reported at 6 weeks. In stark contrast, Lubritz et al. (J. Am. Acad. Dermatol. 1982; 7(5):631-632) reported markedly less recurrence (14% during a follow-up period of 1 to 8.5 years). The freeze time was not given in the latter study and the description of the material and methods is less than optimal. One difference between the trials was that Lubritz et al. looked at the recurrence of treated lesions whereas Krawchenko et al. looked at recurrence and de novo AKs in a field in which individual AKs had been frozen. Tan et. al., (J. Cutan. Med. Surg.; 2007 November-December; 11(6):195-201) illustrates that the difference is due to the development of new AKs in the treatment area. Whereas complete clearance of all target lesions after cryotherapy of a 50 cm² treatment area at Week 22 was reported in 38% of patients, the field was only free of AKs in 9%. In conclusion it is difficult to foresee the rate of recurrence after cryotherapy in the planned study. It seems clear however that de novo lesions will appear in addition to recurrences of treated lesions within a defined treatment area.

Although short term efficacy with cryotherapy demonstrates good clearance of individual lesions, recurrence rates are high. In addition, lesion directed treatments such as cryotherapy, fail to address the issue of field cancerisation in patients with AK. Results from the Phase 3 studies of ingenol mebutate (PEP005 Gel) report complete field clearance of 37-47% on the face and scalp and sustained clearance around 50% at 12 month following treatment.

It is an object of the present invention to provide a treatment of actinic keratosis lesions, which provides a field directed treatment in an area surrounding the actinic keratosis lesions. It is an object of the invention to improve the clearance rate of subclinical actinic keratosis lesions in the treated field.

It is an object of the present invention to provide a method for treating actinic keratosis which improves the clearance rate of the actinic keratosis lesions in the treated area compared to cryotherapy alone.

It is an object of the present invention to provide a method for treating actinic keratosis which improves the partial clearance of the actinic keratosis lesions in the treated area compared to cryotherapy alone.

It is an object of the present invention to provide a method for treating actinic keratosis which reduces the number of actinic keratosis lesions in the treated area compared to cryotherapy alone.

### SUMMARY OF THE INVENTION

The invention provides a method for treating actinic keratosis in subject in need thereof comprising a combination of cryotherapy and topical treatment with ingenol mebutate.

The invention provides a method according to above, comprising applying cryotherapy to the lesion, followed by topical application of ingenol mebutate.

The invention provides a method according to the above, wherein the topical treatment with ingenol mebutate is started 14-31 days after cryotherapy of the lesion.

The invention provides a method according to any of the embodiments above, wherein the ingenol mebutate is applied as a field therapy covering maximum of 25 cm².

The invention provides a method according to any of the embodiments above, wherein the subject is treated with 0.015% ingenol mebutate on face or scalp for 3 consecutive days.

The invention provides a method according to any of the embodiments above, wherein the subject is treated with 0.05% ingenol mebutate on trunk and extremities, for 2 consecutive days.

In an embodiment of the invention, complete clearance of AK lesions in the treated area of the skin of the subject, defined as the proportion of subjects with no clinically visible AK lesions in the selected treatment area, is observed after the combination treatment.

In an embodiment of the invention complete clearance of AK in the treated area of the skin of the subject, defined as no clinically visible AK's, is observed at week 11 after treatment start and until 12 months from treatment start.

In an embodiment of the invention, the number of clinically visible AK lesions in the treated area of the skin of the subject is reduced.

It is an object of the present invention to improve the rate of complete clearance of AKs using sequential cryotherapy and field treatment with PEP005 Gel compared to cryotherapy alone.

It is an object of the present invention to reduce the rate of recurrence in the treated area compared to cryotherapy alone. In an embodiment the invention provides ingenol mebutate for treating actinic keratosis in subject in need thereof, comprising administering a combination of cryotherapy and topical treatment with ingenol mebutate to the subject.

In an embodiment the invention provides ingenol mebutate for treating actinic keratosis in subject in need thereof, comprising applying cryotherapy to an actinic keratosis lesion on the skin of the subject, followed by topical application of ingenol mebutate to the skin of the subject.

In an embodiment of the invention according to any of the embodiments above, the topical application of ingenol mebutate is started 14-31 days after the application of cryotherapy to the actinic keratosis lesion.

In an embodiment of the invention according to any of the embodiments above, the ingenol mebutate is applied as a field therapy covering maximum of 25 cm².

In an embodiment of the invention according to any of the embodiments above, the subject is treated with 0.015% ingenol mebutate on face or scalp for 3 consecutive days.

In an embodiment of the invention according to any of the embodiments above, the subject is treated with 0.05% ingenol mebutate on trunk and extremities, for 2 consecutive days.

In an embodiment of the invention according to any of the embodiments above, the topical treatment with ingenol mebutate comprises topical treatment with a pharmaceutical formulation of ingenol mebutate.

In an embodiment of the invention according to any of the embodiments above, pharmaceutical formulation of ingenol mebutate comprises ingenol mebutate and a pharmaceutically acceptable carrier.

In an embodiment of the invention according to any of the embodiments above, the pharmaceutical formulation of ingenol mebutate is a gel.

In an embodiment of the invention according to any of the embodiments above, the pharmaceutical formulation of ingenol mebutate comprises 0.015% ingenol mebutate.

In an embodiment of the invention according to any of the embodiments above, wherein complete clearance of actinic keratosis lesions in the treated area of the skin of the subject is achieved.

In an embodiment of the invention according to any of the embodiments above, wherein partial clearance of actinic keratosis lesions in the treated area of the skin of the subject is achieved.

In an embodiment of the invention according to any of the embodiments above, wherein the number of actinic keratosis lesions in the treated area of the skin of the subject is reduced.

In an embodiment the invention provides the use of ingenol mebutate for the manufacture of a medicament for the treatment of actinic keratosis in subject in need thereof, to be used in combination with cryotherapy.

In an embodiment the invention provides the use as above comprising applying cryotherapy to an actinic keratosis lesion on the skin of the subject, followed by topical application of ingenol mebutate to the skin of the subject.

In an embodiment the invention provides the use according to any of the embodiments above, wherein the topical application of ingenol mebutate is started 14-31 days after the application of cryotherapy to the actinic keratosis lesion.

In an embodiment the invention provides the use according to any of the embodiments above, wherein the ingenol mebutate is applied as a field therapy covering maximum of 25 cm².

In an embodiment the invention provides the use according to any of the embodiments above, wherein the subject is treated with 0.015% ingenol mebutate on face or scalp for 3 consecutive days.

In an embodiment the invention provides the use according to any of the embodiments above, wherein the subject is treated with 0.05% ingenol mebutate on trunk and extremities, for 2 consecutive days.

In an embodiment the invention provides the use according to any of the embodiments above, wherein the topical treatment with ingenol mebutate comprises topical treatment with a pharmaceutical formulation of ingenol mebutate.

In an embodiment the invention provides the use according to any of the embodiments above, wherein the pharmaceutical formulation of ingenol mebutate comprises ingenol mebutate and a pharmaceutically acceptable carrier.

In an embodiment the invention provides the use according to any of the embodiments above, wherein the pharmaceutical formulation of ingenol mebutate is a gel.

In an embodiment the invention provides the use according to any of the embodiments above, wherein the pharmaceutical formulation of ingenol mebutate comprises 0.015% ingenol mebutate.

In an embodiment the invention provides the use according to any of the embodiments above, wherein complete clearance of actinic keratosis lesions in the treated area of the skin of the subject is achieved.

In an embodiment the invention provides the use according to any of the embodiments above, wherein partial clearance of actinic keratosis lesions in the treated area of the skin of the subject is achieved.

In an embodiment the invention provides the use according to any of the embodiments above, wherein the number of actinic keratosis lesions in the treated area of the skin of the subject is reduced.

### BRIEF DESCRIPTION OF THE DRAWINGS

FIG. 1 is a graphical illustration of the study design described in Example 2.

FIG. 2 is a graph showing the rates of complete clearance and partial clearance after treatment with cryotherapy and ingenol mebutate versus cryotherapy with a vehicle control.

FIG. 3 shows AK lesion counts for patients after treatment with cryotherapy and ingenol mebutate versus cryotherapy with a vehicle control.

FIG. 4 is a graph showing composite local skin reaction (LSR) score after treatment with cryotherapy and ingenol mebutate versus cryotherapy with a vehicle control.

### DETAILED DESCRIPTION OF THE INVENTION

In an embodiment, the short-term (2-3 months) complete clearance rate of cryotherapy-treated AKs as well as the treated field after the sequential cryotherapy and field treatment using ingenol mebutate (PEP005 Gel), is superior to cryotherapy alone.

In an embodiment the short term (2-3 months) partial clearance rate of cryotherapy-treated AKs as well as the treated field after the sequential cryotherapy and field treatment using ingenol mebutate (PEP005 Gel), is superior to cryotherapy alone.

In an embodiment, the short-term (2-3 months) reduction in number of AKs as well as the treated field after the sequential cryotherapy and field treatment using ingenol mebutate (PEP005 Gel), is superior to cryotherapy alone.

In an embodiment the long-term (12 months) complete clearance rate of cryotherapy-treated AKs as well as the treated field after the sequential cryotherapy and field treatment using ingenol mebutate (PEP005 Gel), is superior to cryotherapy alone.

In an embodiment the long term (12 months) partial clearance rate of cryotherapy-treated AKs as well as the treated field after the sequential cryotherapy and field treatment using ingenol mebutate (PEP005 Gel), is superior to cryotherapy alone.

In an embodiment the long term recurrence rate in the treated area is reduced compared to cryotherapy alone.

In the context of the present invention partial clearance of AKs are defined as 75% or greater reduction in the number of clinically visible AKs in the selected treatment area from baseline.

Cryotherapy of AK lesions can be performed according to methods known in the art. For example, liquid nitrogen can be applied to an AK lesion to freeze and remove the lesion. Compressed nitrous oxide or carbon dioxide can also be used. The cryogen (e.g. liquid nitrogen) is applied to an AK lesion for a freeze time long enough to freeze and preferably remove the AK lesion, e.g., from about five seconds to about one minute, more preferably from about five seconds to about 30 seconds. Longer freeze times are generally associated with higher response rates than shorter freeze times. Cryotherapy can be applied to 1, 2, 3, 4, 5, 6, 7, 8, 9, 10 or more AK lesions.

In general, any pharmaceutical formulation of ingenol mebutate that is suitable for topical administration can be used in the method of the invention. Pharmaceutical formulations of ingenol mebutate include an effective amount of ingenol mebutate and a pharmaceutically acceptable carrier (including solvents, excipients, fillers, and the like).

The following exemplary ingenol mebutate formulations can be used in the methods of the invention:

PEP005 gel 0.015% and 0.05% contains 150 mcg and 500 mcg of ingenol mebutate, respectively, in each gram of gel consisting of isopropyl alcohol, hydroxyethyl cellulose, citric acid monohydrate, sodium citrate, benzyl alcohol and purified water. Gel is a clear colorless gel and supplied in unit dose laminate tubes, for single use, containing a nominal fill weight of 0.47 g, with a deliverable weight of 0.25 g. The tubes should be discarded after a single use.

### EXAMPLE

### Study Design

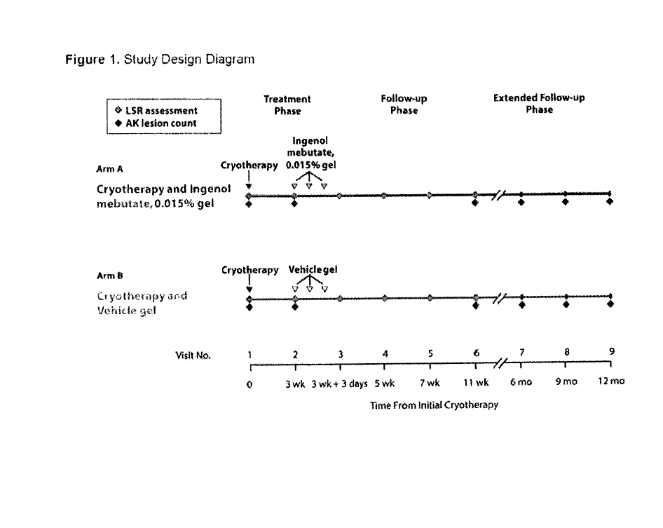

A Phase 3, multi-centre, randomised, two-arm, parallel group, double-blinded, vehicle-controlled, 12-month study is conducted (See FIG. 1). Eligible subjects with 4 to 8 clinically typical, visible and discrete AKs within a contiguous 25 cm² treatment area on the face and scalp will be randomised to one of two arms. Treatment Arm A will receive cryotherapy to all visible AKs (4-8 lesions, “baseline lesions”) in the selected treatment area then, after 2-4 weeks healing time, field treatment with ingenol mebutate (PEP005 Gel) 0.015% once daily for 3 consecutive days. Treatment Arm B will receive cryotherapy to all visible AKs (4-8 lesions, “baseline lesions”) in the selected treatment area then, after 3 weeks healing time, field treatment with vehicle gel once daily for 3 consecutive days.

Eligible subjects include male or female, at least 18 years of age, with 4 to 8 clinically typical, visible, and discrete AK lesions within a 25 cm² contiguous treatment area on the face or scalp. Exclusion criteria stipulate restrictions for prohibited treatments and procedures prior to study entry. This sample is representative of the population which will be treated for AK.

A two-arm trial has been chosen with cryotherapy plus PEP005 as a sequential treatment in one arm and cryotherapy plus vehicle in the other arm. The study has almost identical inclusion and exclusion criteria and will be conducted in the same geographical area as the pivotal/confirmatory phase 3 trials which were finalised recently, and enrolled approximately 500 patients. Therefore it will be possible to compare the efficacy and the safety of the new trial directly with the data of the previous phase 3 trials.

Cryotherapy followed by trial medication (PEP005 Gel, 0.015% or vehicle gel) was chosen as the optimal sequence because once field treatment has been applied it would be difficult to aim the lesion-specific cryotherapy at the lesions, especially if the initial PEP005 Gel treatment had diminished or eradicated the AK. Additionally, the established clinical practice in the US is to administer cryotherapy first, sometimes followed weeks later by the addition of a topical product such as 5-fluorouracil.

Randomisation is stratified by study site and by location of the selected treatment area (face or scalp). The percentage of scalp and face treated subjects will be controlled so that it represents the population which will be treated. Approximately 80% of subjects enrolled will be treated on the face and 20% will be treated on the scalp. This ratio was selected based on published survey results which tabulated the anatomical location of AK lesions in patients seeking treatment for AK. A double-blinded, parallel group design has been selected because the purpose is to investigate efficacy with comparisons between the treatment arms. This design provides an unbiased assessment of data.

For safety, AEs and LSRs will be recorded. Local skin responses will be graded using a scale that was developed by the sponsor and used in the previous clinical studies. This is a defined grading scale to ensure that a clear and systematic assessment of LSRs is performed. The LSR Grading Scale employs a 0 to 4 scoring system for each category to be assessed with photographs and definitions of each grade. The LSR Grading Scale has been published previously.

The incidence and grade of LSRs in the LSR safety set will be summarized by treatment arm overall and at each visit by anatomical location. Local skin response grades will be summarized by frequency counts and descriptive statistics by treatment arm and visit for each of the six individual LSRs: erythema, flaking/scaling, crusting, swelling, vesiculation/pustulation, and erosion/ulceration.

A composite (sum) score will be obtained by summing the six individual LSR scores at each visit. The composite score and change from baseline will be summarized by treatment arm at each visit using descriptive statistics. The first visit at which the highest sum score is attained will be summarized by treatment arm. For those subjects whose LSR sum score increased in intensity from baseline, the first visit at which the LSR returned to the baseline intensity will be presented by treatment arm.

The highest grade post-baseline and incidence of any response (post-baseline) for each LSR type will also be presented by frequency distribution for each treatment arm. The study day of the highest grade greater than baseline for each LSR type and the study day at which the grade returns to a level at or below the baseline level will also be presented by frequency distribution for each treatment arm.

Local skin responses will be converted into MedDRA preferred terms applying the following conversions seen below. These adverse events will be reported separately from adverse events recorded on the adverse event form in the CRF.

Conversion of LSRs to MedDRA Preferred Terms:

Conversion of LSRs to MedDRA Preferred Terms

| LSR Term | LSR Grade | MedDRA Preferred Term |
|---|---|---|
| Erythema | 1-4 | Application site erythema |
| Flaking/Scaling | 1-4 | Application site exfoliation. |
| Crusting | 1-4 | Application site scab |
| Swelling | 1-4 | Application site swelling |
| Vesiculation/ | 1 | Application site blister |
| Pustulation | 2-4 | Application site pustules |
| Erosion/ | 1-3 | Application site erosion |
| Ulceration | 4 | Application site ulcer |

### Exclusion Criteria

1

Skin type of the subjects will be assessed by the investigator and recorded according to the following classification:

### Fitzpatrick Skin Types

I Always burns easily, never tans
II Always burns easily, tans minimally
III Burns moderately, tans gradually (light brown)
IV Burns minimally, always tans well (moderate brown)
V Rarely burns, tans very well (moderate brown)
VI Never burns, deeply pigmented

### Example

A Phase 3, multi-centre, randomised, two-arm, parallel group, double-blinded, vehicle-controlled, 12-month study is being conducted (See FIG. 1). Eligible subjects with 4 to 8 clinically typical, visible and discrete AKs within a contiguous 25 cm² treatment area on the face and scalp were randomised to one of two arms. Treatment Arm A received cryotherapy to all visible AKs (4-8 lesions, “baseline lesions”) in the selected treatment area then, after 2-4 weeks healing time, they received field treatment with ingenol mebutate (PEP005 Gel) 0.015% once daily for 3 consecutive days. Treatment Arm B received cryotherapy to all visible AKs (4-8 lesions, “baseline lesions”) in the selected treatment area then, after 3 weeks healing time, they received field treatment with vehicle gel once daily for 3 consecutive days.

Eligible subjects included male or female, at least 18 years of age, with 4 to 8 clinically typical, visible, and discrete AK lesions within a 25 cm² contiguous treatment area on the face or scalp. Exclusion criteria stipulated restrictions for prohibited treatments and procedures prior to study entry. This sample was representative of the population which will be treated for AK.

Cryotherapy followed by trial medication (PEP005 Gel, 0.015% or vehicle gel) was applied according to the trial design. Randomisation was stratified by study site and by location of the selected treatment area (face or scalp). The percentage of scalp and face treated subjects was controlled so that it represents the population which will be treated. In the present study 80.5% of subjects enrolled was treated on the face and 19.5% was treated on the scalp.

For safety, AEs and LSRs were recorded. Local skin responses were graded using a scale that was developed by the sponsor and used in the previous clinical studies. This was a defined grading scale to ensure that a clear and systematic assessment of LSRs is performed. The LSR Grading Scale employs a 0 to 4 scoring system for each category to be assessed with photographs and definitions of each grade.

### Results:

329 patients were enrolled displaying the following characteristics:

TABLE 1
Characteristic

| Fitzpatrick skin type | patients |
|---|---|
| I | 15.2% |
| II | 48.0% |
| III | 27.7% |
| IV, V | 9.1% |
| Male sex | 82.4% |
| Treatment location | |
| Face | 80.5% |
| Scalp | 19.5% |
| Age | |
| Median (range) | 67.0 (34-89) years |
| History of skin cancer | 47.1% |
| Duration of AK | |
| Median (range) | 10.3 (0-55) years |
| AK treatment history | |
| Cryosurgery | 89.1% |
| 5-Fluorouracil | 18.5% |
| Imiquimod | 17.3% |
| Surgical excision | 15.5% |
| Photodynamic therapy | 14.6% |
| Other treatments | 18.8% |
| Any previous treatment | 94.2% |

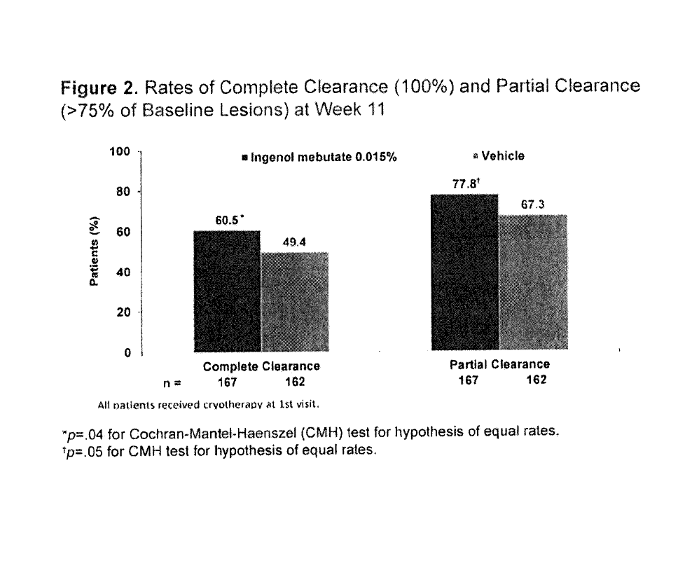

The primary efficacy analysis of complete clearance at week 11 (defined as no clinically visible AKs) was the comparison of the 2 treatment arms using a CMH chi-square test stratified by anatomical location and study site with significance level of 5%. The percentage of patients with complete clearance was higher in the ingenol mebutate group than in the vehicle group (60.5% vs 49.4%) (p=0.04) (FIG. 2). The percentage of patients with partial clearance was higher in the ingenol mebutate group than in the vehicle group (77.8% vs 67.3%) (p=0.05) (FIG. 2). Baseline lesion count was similarly distributed across the groups.

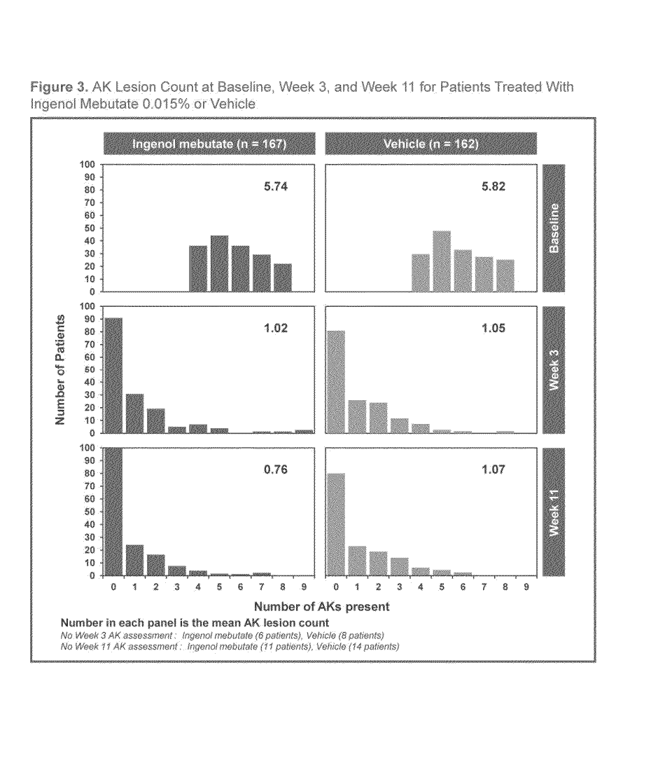

A general reduction from baseline lesion count was observed 3 weeks after cryosurgery, and few AKs remained to be cleared by topical treatment (FIG. 3).

At week 11, AK lesion count was lower in the ingenol mebutate group as measured by Median* percent reduction from baseline:

Ingenol mebutate: 100.0%
Vehicle: 87.5%. *Medians of 100% are produced when complete clearance rates are greater than 50% (shown in FIG. 3)

The treatment was well tolerated by the patients as indicated by adverse effects shown below. The most common adverse effect is application site pain. None of the severe adverse events were related to trial medication. All adverse effects related to trial medication were resolved at week 11.

TABLE 2
AEs Related to Trial Medication by MedRA System Organ Class

| | |
|---|---|
| Ingenol mebutate gel, | Vehicle gel |
| 0.015% (n = 167) | (n = 162) |
| No. | No. |

System organ class preferred term*
Cardiac

| | | |
|---|---|---|
| Tachycardia | 1 | 0 |

Eye disorders

| | | |
|---|---|---|
| Eye irritation | 1 | 0 |
| Eye swelling | 2 | 0 |
| Lacrimation increased | 1 | 0 |

Gastrointestinal disorders

| | | |
|---|---|---|
| Nausea | 1 | 0 |

General disorders and administration site conditions

| | | |
|---|---|---|
| Application-site discoloration | 0 | 1 |
| Application-site induration | 1 | 0 |
| Application-site pain | 7 | 0 |
| Application-site pruritus | 3 | 0 |
| Swelling | 1 | 0 |

Injury, poisoning, and procedural complications

| | | |
|---|---|---|
| Chemical eye injury | 1 | 0 |

Nervous system disorders

| | | |
|---|---|---|
| Headache | 3 | 0 |

Skin and subcutaneous tissue disorders

| | | |
|---|---|---|
| Dermatitis | 1 | 0 |
| Skin tightness | 2 | 0 |
| Total number of AEs¹ | 25 | 1 |
| Total number of patients | 19 | 1 |

*MedRA version 14.1.
¹Different adverse events within the same preferred term and system organ class and involving the same subject have been counted as 1 adverse event. An individual patient could appear in multiple classes.

With respect to LSRs, at baseline the composite LSR score was similar in both groups (ingenol mebutate: 2.2; vehicle, 2.1). After cryosurgery, the composite LSR score decreased slightly in both groups over the 3-week period prior to topical field treatment.

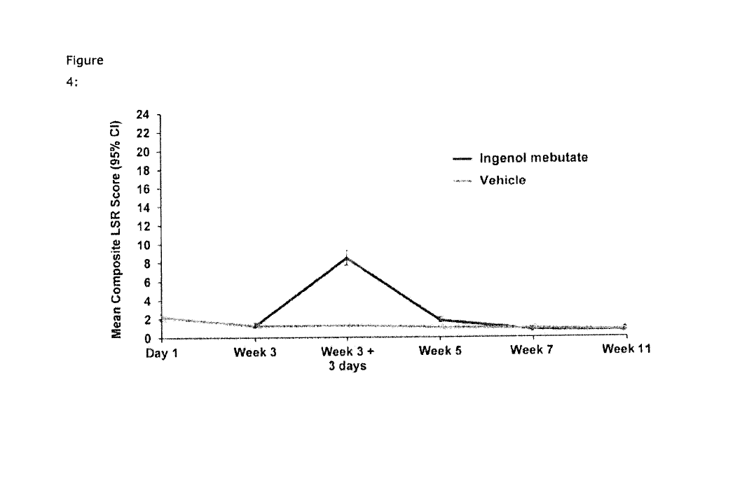

The mean (SD) composite LSR score in the ingenol mebutate group increased and peaked at visit 3 at 8.5 (4.7) (FIG. 4).

At week 5, 2 weeks after topical treatment, mean composite LSR score in the ingenol mebutate group return to a score similar to that of earlier visits (results shown in FIG. 4).

TABLE 3
Maximum Composite LSR Score for
All Patients, Safety Population*

| | Ingenol mebutate gel, | Vehicle gel |
|---|---|---|
| LSR score | 0.015% (n = 157) | (n = 150) |
| Mean (SD) | 8.5 (4.7) | 1.5 (1.4) |
| Median (range) | 8.0 (0-20) | 2.0 (0-6) |

*At any time after week 3.

### CONCLUSIONS

These results show that:

(1) Short-term (11-week) clearance rates of AKs on the face or scalp with ingenol mebutate 3 weeks after cryosurgery were higher than with cryosurgery alone.
(2) Despite effective clearance of AKs by cryosurgery, the complete clearance rate (CCR) in the group that received ingenol mebutate (60.5%) after cryosurgery was significantly higher than the CCR for cryosurgery alone (49.4%).
(3) Treatment with ingenol mebutate after cryosurgery was well tolerated. LSRs that occurred with ingenol mebutate treatment were similar to LSRs reported in other phase 3 studies of ingenol mebutate (e.g., Lebwohl M, Swanson N, Anderson L L, et al. Ingenol mebutate gel for actinic keratosis. N Engl J Med. 2012; 366:1010-1019) with respect to severity, time of onset, and time of resolution.
(4) No safety concerns were identified.
(5) The data obtained at week 11 indicates that ingenol mebutate treatment after cryosurgery improves short-term clearance rates and is safe and tolerable on the face or scalp.

1. A method for treating actinic keratosis in a subject in need thereof, the method comprising administering a combination of cryotherapy and topical treatment with ingenol mebutate to the subject.

2. The method according to claim 1, comprising applying cryotherapy to an actinic keratosis lesion on the skin of the subject, followed by topical application of ingenol mebutate to the skin of the subject.

3. The method according to claim 2, wherein the topical application of ingenol mebutate is started 14-31 days after the application of cryotherapy to the actinic keratosis lesion.

4. The method according to claim 1, wherein the ingenol mebutate is applied as a field therapy covering maximum of 25 cm².

5. The method according to claim 1, wherein the subject is treated with 0.015% ingenol mebutate on face or scalp for 3 consecutive days.

6. The method according to claim 1, wherein the subject is treated with 0.05% ingenol mebutate on trunk and extremities, for 2 consecutive days.

7. The method of claim 1, wherein the topical treatment with ingenol mebutate comprises topical treatment with a pharmaceutical formulation of ingenol mebutate.

8. The method of claim 7, wherein the pharmaceutical formulation of ingenol mebutate comprises ingenol mebutate and a pharmaceutically acceptable carrier.

9. The method of claim 7, wherein the pharmaceutical formulation of ingenol mebutate is a gel.

10. The method of claim 1, wherein the pharmaceutical formulation of ingenol mebutate comprises 0.015% ingenol mebutate.

11. The method of claim 1, wherein the method results in complete clearance of actinic keratosis lesions in the treated area of the skin of the subject.

12. The method of claim 1, wherein the number of clinically visible AK lesions in the treated area of the skin of the subject is reduced after treatment.
